(19) United States
(12) Patent Application Publication
(10) Pub. No.: US 2016/0302658 A1
(43) Pub. Date: Oct. 20, 2016

(54) FILTERING EYE BLINK ARTIFACT FROM INFRARED VIDEONYSTAGMOGRAPHY

(71) Applicant: Marcello Cherchi, Lincolnwood, IL (US)

(72) Inventor: Marcello Cherchi, Lincolnwood, IL (US)

(21) Appl. No.: 14/689,209

(22) Filed: Apr. 17, 2015

(51) Int. Cl. A61B 3/00; A61B 3/14; A61B 5/00; A61B 3/113

(52) U.S. Cl. CPC A61B 3/0025; A61B 3/113; A61B 3/145; A61B 5/7246; A61B 5/7253; A61B 3/0041

(57) ABSTRACT

As provided in accordance with the present invention there is provided a simple, effective algorithm for filtering out eye blink artifact at the level of individual grayscale images commonly acquired for medical diagnostic purposes by infrared videonystagmography.

### BACKGROUND OF THE INVENTION

Observation of eye movements is important in the fields of neurology, otolaryngology and audiology for diagnosis of vestibular disorders (disturbances of equilibrium). This can be accomplished at a basic, qualitative level through physical examination by a physician, but it is desirable to record this information in a systematic fashion for purposes of quantification, storage, retrieval and comparison. Computerized analysis of eye movements is a well-developed technology.

Early technology for recording eye movements included electro-nystagmography (also called electro-oculography), though this approach had a number of drawbacks. The advent of videonystagmography (VNG), sometimes also referred to as video-oculography (VOG), essentially involves recording a video of one eye (or each eye in separate video streams), determining the pupil's center in each video frame, and plotting the X and Y coordinates of the pupil's center over time, thereby generating a “tracing” of horizontal and vertical components of eye movements. These tracings (the actual videonystagmograms) can aid in diagnosis when analyzed properly.

While videonystagmography has been fairly successful, a significant limitation has been found in the distortion of the tracing by artifact, of which by far the most copious and intrusive is that introduced by eye blinks. Eye blink artifact often results in a tracing that, during the eye blinks, may falsely appear to record movement of the pupil, and can easily lead to a variety of “false positive” diagnostic errors. There are methods that offer much greater temporal and spatial resolution and can avoid eye blink artifact entirely, such as the scleral search coil technique but this technique is largely confined to research settings, as it is too cumbersome for routine clinical use, and would be impractical in the setting of acute medical care. As such the existing approaches to filtering eye blink artifact are not very effective.

Subspecialist physicians review not just the nystagmographic tracing, but often also the original eye movement video, which enables them to recognize directly any eye blink artifact. However, the use of videonystagmography is expanding beyond the subspecialist domain, and current research and practice trends suggest that this technology will soon be deployed in the emergency room setting to help diagnose patients with acute vestibular disorders and make real-time medical management decisions. In this context, in which non-subspecialty physicians (who do not directly review the raw videos) attempt to use this technology, it is anticipated that the interpretation of eye movement tracings will be heavily computer-driven. In order to increase the diagnostic accuracy of a computer's analysis of an eye movement tracing, it is essential to provide the computer with data that are as “clean” as possible, and that goal will be advanced by effective filtering of eye blink artifact.

A need therefore exists for software to effectively filter the eye blink artifact from infrared videonystagmography. The present invention includes software that operates at the level of individual video frame images by leveraging simple properties of the Hough transform accumulator matrix for shape (circle) recognition in order to identify when the pupil is detected (corresponding to the eye being open) or not (corresponding to when the eye is in the midst of a blink). The data indicate that the software, when implemented against real clinical examples (i.e., not from an idealized dataset), performs better than a commonly used commercial software package.

### SUMMARY OF THE INVENTION

In accordance with one or more embodiments of the present invention there is provided a system for videonystagmography (VNG) testing of a pupil in an eye that records oculomotor response data and has a computing device configured with software to determine and display on a display device a plot representation of the correlated data. An improvement to the software instructions that determine pupil recognition and plot representation of the correlated data includes instructions configured to (a) extract a grayscale image from a frame in the recorded oculomotor response data; (b) locate and identify an edge of a shape from the extracted image of the eye, referred to as an identified shape edge; (c) determine a center and a diameter from the identified shape edge and store the diameter in a range from smallest to largest probable diameters; (d) run a shape identification Hough transform on the identified shape edge, identifying a shape representing the candidate pupil in the extracted image, wherein the Hough transform iterates from the smallest probable diameter to the largest probable diameter and the software renders an accumulator matrix; (e) compare a current amplitude of the center and the diameter of the candidate shape to an average of amplitudes of other candidate shapes defined by the accumulator matrix, which defines an absolute amplitude of the center and the diameter and defines an average-to-peak ratio; (f) compare the absolute amplitude and the average-to-peak ratio to two threshold criterion parameters to determine the likelihood of pupil recognition; (g) plot coordinates representation of the center of the candidate shape only when the candidate shape meets the two threshold criterion parameters for pupil recognition.

In other aspects of the embodiments, the software may be further configured to identify an adjusted edge shape from the identified shape edge based on an adjustable threshold algorithm. In addition, the software may be further configured to compare the adjusted shape edge in the extracted image to a previous identified shape edge in a previous extracted image to determine the shape diameter. Yet in further embodiments, the software may be further configured to run the shape identification Hough transform on the adjusted shape edge to identify the candidate shape and to render the Hough accumulator matrix.

The software is developed to advance to the subsequent frame in the recorded oculomotor response data without plotting the center of the shape coordinates when the candidate shape fails to meet the two threshold criterion parameters for pupil recognition. Similarly, the software is designed to repeat the process for each frame in the recorded oculomotor response data.

In the present embodiments, the shape identification transform may be based on either a circular identification transform or an elliptical identification transform.

In one or more other embodiments, a first threshold criterion parameter may be an absolute amplitude of the peak of the candidate shape and is configured to a low acceptable value to define a less stringent criterion for pupil identification or is configured to a high acceptable value to define a more stringent criterion for pupil identification. Further expanding on the threshold criterion parameters, a second threshold criterion parameter may be the average-to-peak ratio and may be configured as a best-fit-circle to quantify a candidate shape and wherein a low average-to-peak ratio is configured for a more stringent criterion for pupil identification and wherein a high average-to-peak ratio is configured for a less stringent criterion for pupil identification.

Numerous other advantages and features of the invention will become readily apparent from the following detailed description of the invention and the embodiments thereof, from the claims, and from the accompanying drawings.

### BRIEF DESCRIPTION OF THE DRAWINGS

A fuller understanding of the foregoing may be had by reference to the accompanying drawings, wherein:

FIG. 1 is a block diagram showing the filtering system process according to the prior art;

FIG. 2 illustrates the use of the prior art Kalman filter to eliminate eye blink artifact from raw tracing data;

FIG. 3 illustrates the tracing data when using a best fit algorithm as provided in the prior art;

FIG. 4 illustrates videonystagmogram tracing in the prior art with copious eye blinks, and no “de-blink” filter;

FIG. 5 illustrates Videonystagmogram tracing in the prior art with copious eye blinks, and with “de-blink” filter applied;

FIG. 6A-6K illustrate several prior art examples of diagnostic errors resulting from software misinterpretation of eye blink artifacts;

FIG. 7 illustrates an example of correct recognition when a pupil has been identified;

FIG. 8 illustrates an example of correct recognition when no pupil has been identified;

FIG. 9 illustrates an algorithm completely filtering out eye blinks;

FIG. 10 illustrates in comparison the performance of a prior art commercial software package against the algorithm in accordance with the present invention;

FIG. 11 illustrates in comparison the performance of a prior art commercial software package against the algorithm in accordance with the present invention;

FIG. 12 illustrates in comparison the performance of a prior art commercial software package against the algorithm in accordance with the present invention;

FIG. 13 illustrates in comparison the performance of a prior art commercial software package against the algorithm in accordance with the present invention;

FIG. 14 illustrates in comparison the performance of a prior art commercial software package against the algorithm in accordance with the present invention;

FIG. 15 illustrates in comparison the performance of a prior art commercial software package against the algorithm in accordance with the present invention;

FIG. 16 illustrates in comparison the performance of a prior art commercial software package against the algorithm in accordance with the present invention;

FIG. 17 illustrates in comparison the performance of a prior art commercial software package against the algorithm in accordance with the present invention;

FIG. 18 illustrates in comparison the performance of a prior art commercial software package against the algorithm in accordance with the present invention;

FIG. 19 illustrates in comparison the performance of a prior art commercial software package against the algorithm in accordance with the present invention;

FIGS. 20-24 illustrates various steps in the algorithm as provided by the present invention

### DETAILED DESCRIPTION OF THE INVENTION

While the invention is susceptible to embodiments in many different forms, there are shown in the drawings and will be described in detail herein the preferred embodiments of the present invention. It should be understood, however, that the present disclosure is to be considered an exemplification of the principles of the invention and is not intended to limit the spirit or scope of the invention and/or claims of the embodiments illustrated.

Quantitative assessment of the eyes (vestibular ocular reflex) (VOR) and other eye movements under various conditions is carried out in a standard battery of tests known as nystagmography. When video technology is used to detect eye movement, it is called videonystagmography (VNG). Testing is usually carried out in a light-obscuring environment in order to minimize the degree to which visual fixation may suppress nystagmus. The equipment used for VNG testing is defined and known in the art, as such the equipment and/or devices are not described in detail or illustrated herein. However, generally the equipment can be defined as providing a goggle-like frame structure configured for securing to a subject's head in a non-relative-motion condition, where the frame structure includes an eye-enclosing, ambient light-excluding housing, and further includes one or more tensive bands extending from the housing and configured to securely grip a portion of the subject's head. One or more image-capture devices is coupled with the housing. The image-capture devices may in one or more embodiments be infrared image-capturing devices. The one or more image-capture devices are configured to obtain, within a darkened environment of the housing, real-time video of eye movement in response to a range of stimuli and testing conditions. Circuitry is operably coupled with the one or more image-capture devices, and the circuitry is further configured to convert the imagery to computer-readable oculomotor response data. A computing device is configured with correlation instructions which, when executed by the computing device, correlate the oculomotor response data and to display to a user a viewable plot representation of the correlated data via a display device operably coupled with the computing structure.

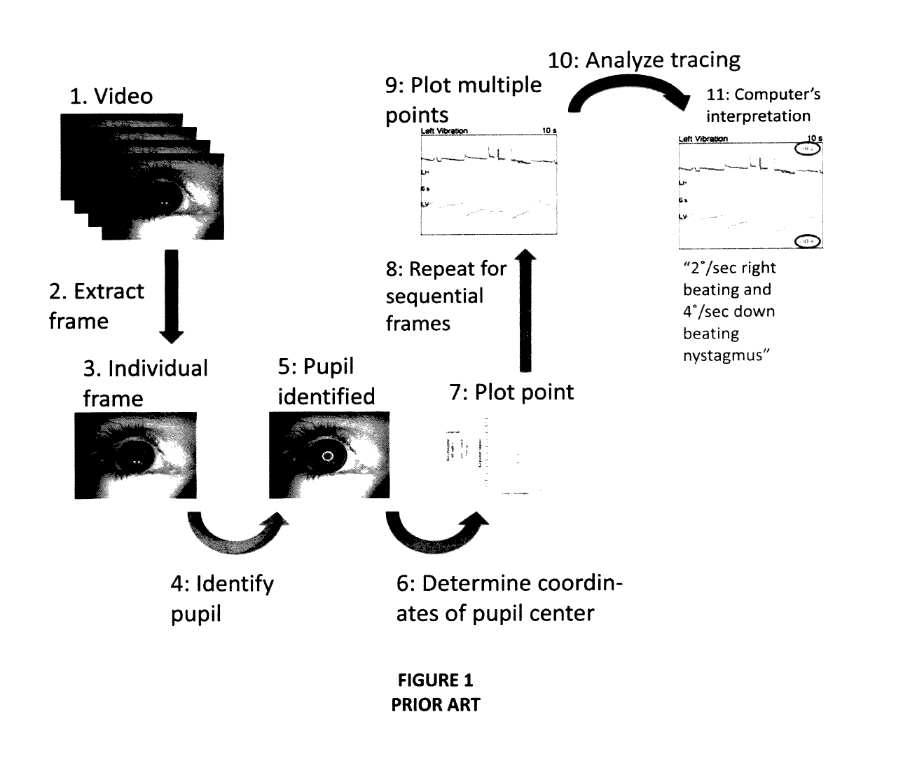

Referring now to FIG. 1 there is shown a summary of the flow of information in videonystagmography and its computerized interpretation as currently provided by the prior art. The steps of this process include Step 1, in which the source video is taken or recorded as data. In Step 2, individual frames are Extracted from the video or data. Each extracted frame, shown in Step 3, is then processed in Step 4 to identify the pupil and its center. Once identified in Step 5, coordinates identifying the pupil's center are determined in Step 6 and then plotted as coordinates in Step 7. In Step 8, Steps 3-7 are repeated for each extract frame and as such a sequence of coordinates over time is generated and then plotted as multiple points in Step 9 to generate raw “tracing” of the data. In Step 10 the tracing is analyzed and in Step 11 the computer system or software interprets the tracing.

The two “processing” steps in this sequence relevant to the current problem are: Step 4 (“Identify pupil and its center”) comprising image recognition that occurs at the level of the individual video frame image; and Step 10 (“Analyze tracing”) comprising pattern recognition in the context of biological signal processing that occurs at the level of the (already generated) tracing of eye movements. The first discussion will focus on filtering at the level of the tracing.

As provided herein, it should be noted that in eye movement tracings, the X-axis corresponds to time and the Y-axis corresponds to the position of the center of the pupil. Typically there are two tracings on a plot; one tracing represents the horizontal component of the eye movement (for which, by convention, upwards on the plot corresponds to a rightward eye movement, and downwards on the plot corresponds to a leftward eye movement), and the other tracing represents the vertical component of the eye movement (for which, by convention, upwards on the plot corresponds to an upward eye movement, and downwards on the plot corresponds to a downward eye movement).

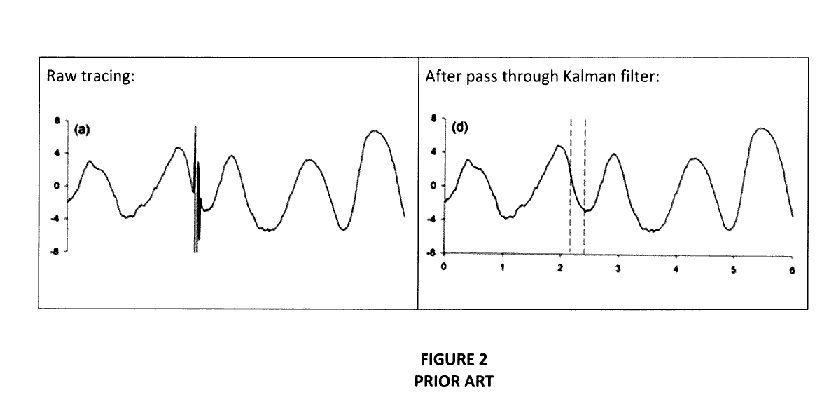

Filtering of eye blink artifact is often performed at the level of the generated tracing. Several techniques have been applied, all of which essentially aim to identify “unexpected” movements, such as movements that are unusual in velocity, magnitude or direction. Relatively rudimentary algorithms simply “clip out” such “unexpected movements.” An example of a more mathematically sophisticated method of accomplishing this is through the use of the known Kalman filter. After “filtering out” such putatively “unexpected movements,” the presumed eye position is then interpolated (and plotted) from the tracing position immediately before the putative artifact, to the tracing position immediately after the putative artifact. An example of this is shown in FIG. 2, which shows the Raw Tracing in box 2A and the Kalman Filtered Results in box 2B.

However, the Kalman Filtering method relies on specific assumptions about what constitutes an “unexpected” eye movement, and in reality, the tracing of apparent movement generated from an eye blink artifact can sometimes be indistinguishable from a true eye movement. Because of this, any attempts to “filter out” eye blinks at the level of the (already generated) tracing are more liable to generate false negative results, in that they may erroneously filter out true eye movements (such as nystagmus). When a system fails to eliminate eye blink artifact, the opposite problem ensues, in that the tracing is much more liable to generate false positive results when analyzed.

In a sequence of processing steps, error in an earlier step is likely propagate downstream and can spawn additional errors in later steps, so a reasonable heuristic is to attempt to catch errors as early as possible in the processing sequence. The implication for the present problem is that blink filtering should be attempted at the level of the individual video frames, before the nystagmographic tracing is even generated.

Numerous publications and patents have proposed a variety of other methods for recognizing eye blinks at the level of the individual frames from the eye movement video, but none has been applied to this specific purpose, nor is any of them likely to be effective in this context. Some examples follow.

Devices applied to the face. Examples include surface electromyogram electrodes, which can detect the electrical activity of muscle contraction that occurs during a blink. Devices that detect reflection of a beam of light. Such approaches aim a beam of light at the eyeball, detect its reflection, and infer an eye blink when that reflection is no longer detectable. Facial feature recognition. This approach employs algorithms that attempt to identify eye blinks in the broader context of facial feature recognition, such as for detecting drowsiness in a driver, or equipping a digital camera such that it does not take a picture when a subject's eyes are closed. Overall luminance threshold. This approach employs algorithms that calculate the overall luminance of a video frame (which should be higher when the eye is open due to the white color of the sclera, and lower when the eye is closed), and infer an eye blink when luminance drops below a predetermined threshold. Frame-to-frame differences. This approach employs algorithms that compare sequential frames and assess specific differences between those frames. Eyelid identification. This approach employs algorithms that attempt to identify the upper eyelid, and infer a blink when the upper eyelid descends below a predetermined threshold or when the velocity of eye movement crosses a specific threshold.

Videonystagmography as applied in clinical use has specific limitations. For instance, much of the examination must be performed with the patient in the dark, because eliminating a patient's ability to fixate visually gives a variety of latent abnormal eye movements (that would otherwise be suppressed by fixation) a greater opportunity to become manifest. In order to accomplish this, infrared illumination is used, and consequently the acquired images are in grayscale, so one cannot exploit differences in hue to distinguish, for instance, the eyelid from the iris or from the sclera. A second limitation is that only the eye and eyelids are visible, and no other portion of the face, which means that methodologies that rely at least in part on facial recognition are not applicable. However, the setting of medical videonystagmography also offers some advantages. For instance, although it is not possible to utilize other facial features, this also means that there are fewer patterns to recognize and therefore a lower burden of computational processing demands, and also means that image resolution is generally higher.

Since the algorithms employed by commercially available software packages for videonystagmographic analysis are proprietary, the software is unavailable for scrutiny. There are however two general strategies for commercially available software packages; one is “blob analysis,” the other is shape (circle or ellipse) recognition.

Blob analysis identifies relatively large contiguous regions in which adjacent pixels have reasonably close luminance—in this case, the sought color is that of the pupil (black). Once such an area has been identified, an algorithm is applied to find the “centroid” of that shape. Identification of the centroid is usually accomplished by determining the weighted average vertical location of adjacent columns (thereby identifying the Y-coordinate of the centroid) and the weighted average horizontal location of adjacent rows (thereby identifying the X-coordinate of the centroid). One of the main difficulties with this technique has to do with identification of the centroid. This can be illustrated with an example. If the eyeball remains in the same position, but the eyelid is half closed such that only the bottom semicircle of the pupil is visible, then the centroid of that visible semicircle will be lower than the actual center of the pupil, even though the pupil itself has not moved. In other words, there will appear to be a downward movement of the eye as a blink occurs.

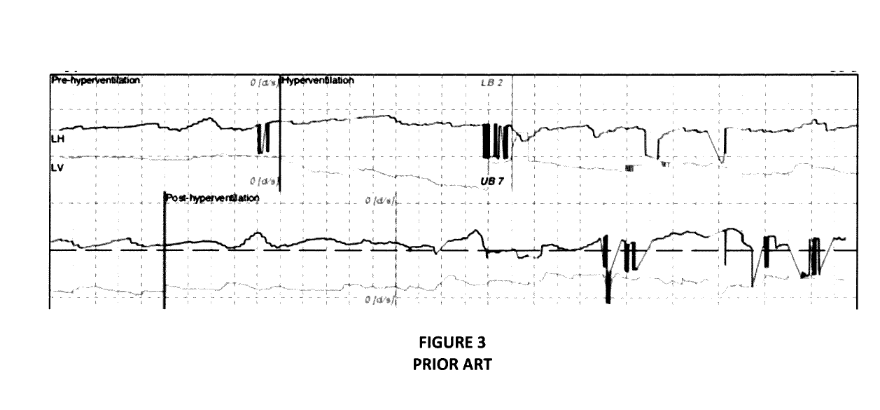

Another approach is that of shape recognition, typically seeking a circle or ellipse. The general approach here is to look for edges in an image (e.g., using Canny edge detection) and then seek a “best-fit” circle or ellipse for those edges (e.g., using a Hough transform). This approach appears sensible because if the pupil can be correctly identified, its center will remain the same even if the circle or ellipse is partly disrupted (e.g., by eyelid occlusion). However, typical shape recognition algorithms are designed to render a “best fit” shape, even if the degree of “fitness” is poor. Consequently if, for instance, a pupil is actually absent in an image (such as when the eyelid is closed), the algorithm will still offer a “best fit” shape, even though the result does not refer to any actual circle or ellipse in the image. The outcome in such circumstances is very messy. The tracing in FIG. 3 using a best fit shape algorithm and has several sections that appear to be closely packed vertical lines; these correspond to times in the video in which the pupil was not visible, and the center of the “best fit” circle was wildly variable, resulting in artifact that should be meaningless, but instead was actually interpreted by this commercial software as representing nystagmus.

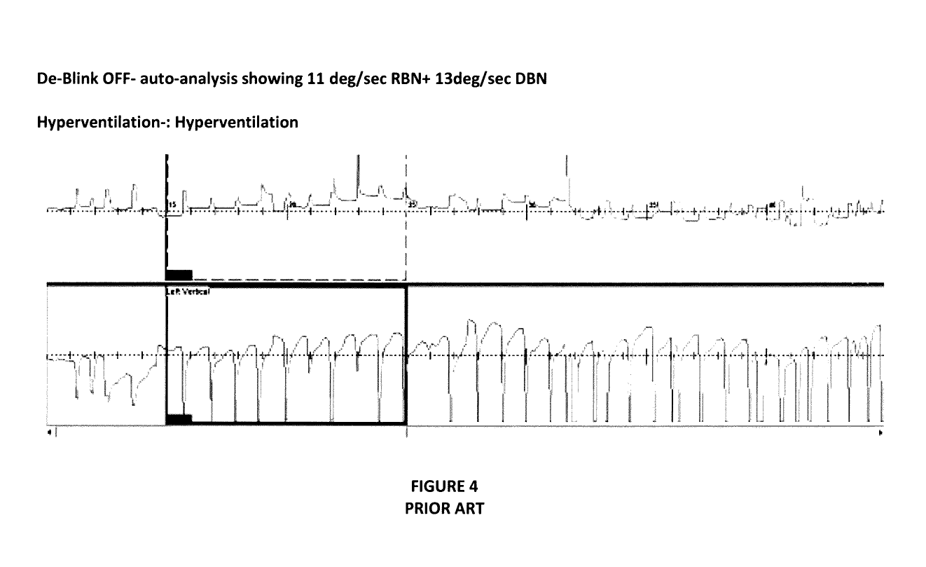

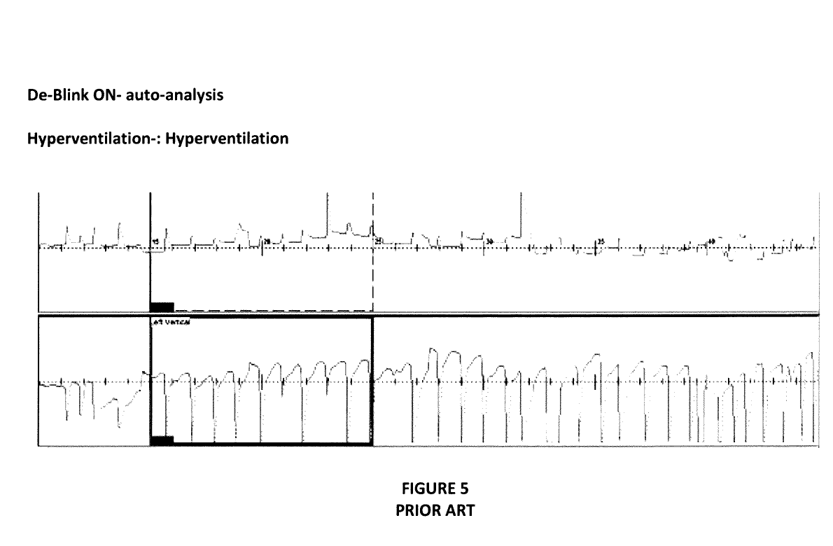

When the most common software package processes an eye movement video with blinking, the resulting tracing appears as in FIG. 4. In the corresponding eye movement video it is clear that the pupil position is relatively stationary; however, in the tracing generated by this software, each eye blink appears as a “spike” that could, in the hands of an inexperienced reader (or someone who does not have access to the original video from which the tracing was derived), be misinterpreted as pathological nystagmus (an abnormal eye movement) and thereby limit the test's diagnostic utility, and even lead to misdiagnosis. This widely used, commercially available software package claims to have a “de-blink” filter to address this problem. When that filter is applied, the result appears as in FIG. 5.

Comparing FIG. 5 (in which the “de-blink” filter is turned on) to FIG. 4 (in which the “de-blink” filter is turned off), it is evident that the “de-blink” filter in this widely-used, commercially available software package, hardly filters out anything at all; it is ineffective; and the computer misinterprets these spikes as nystagmus.

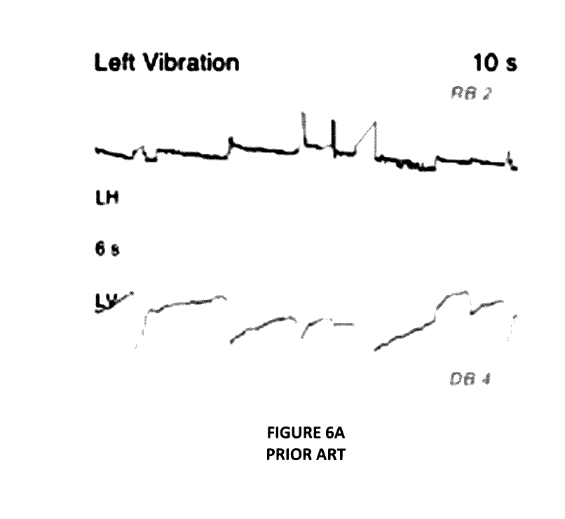

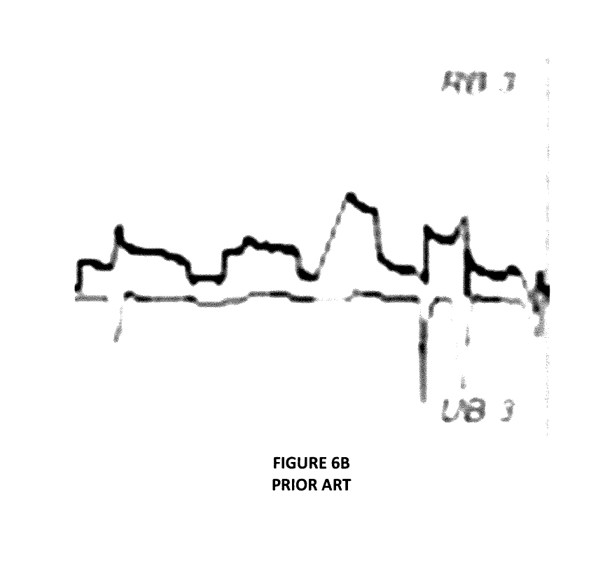

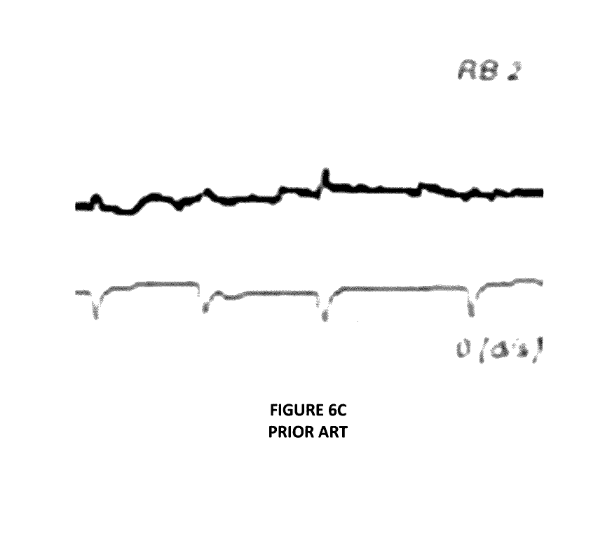

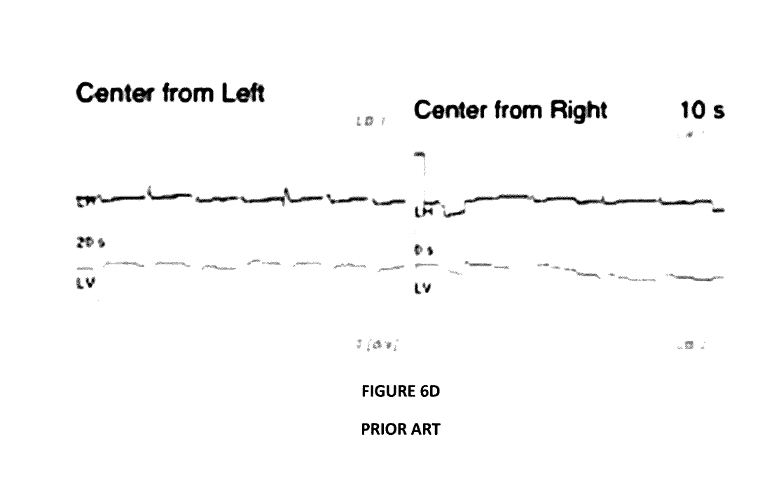

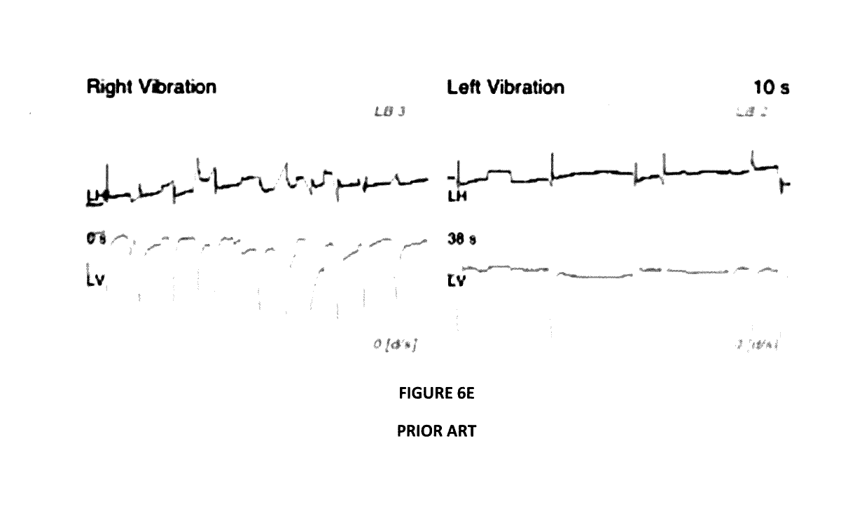

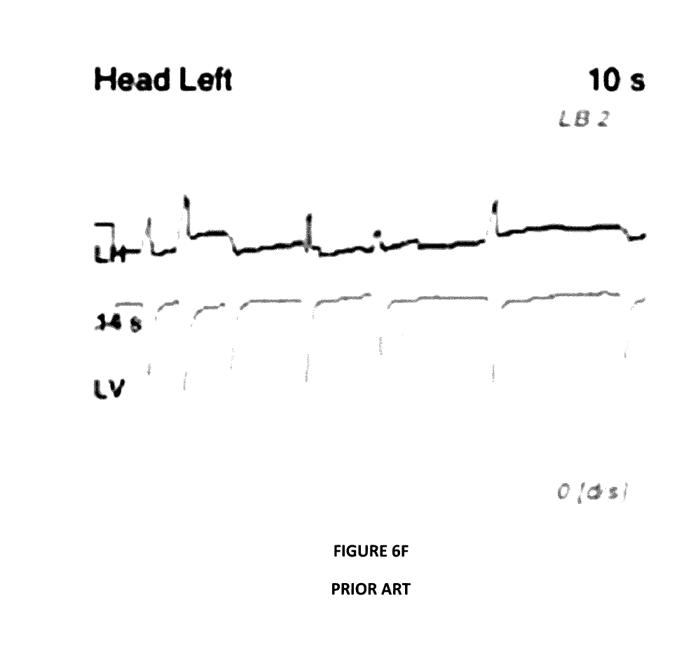

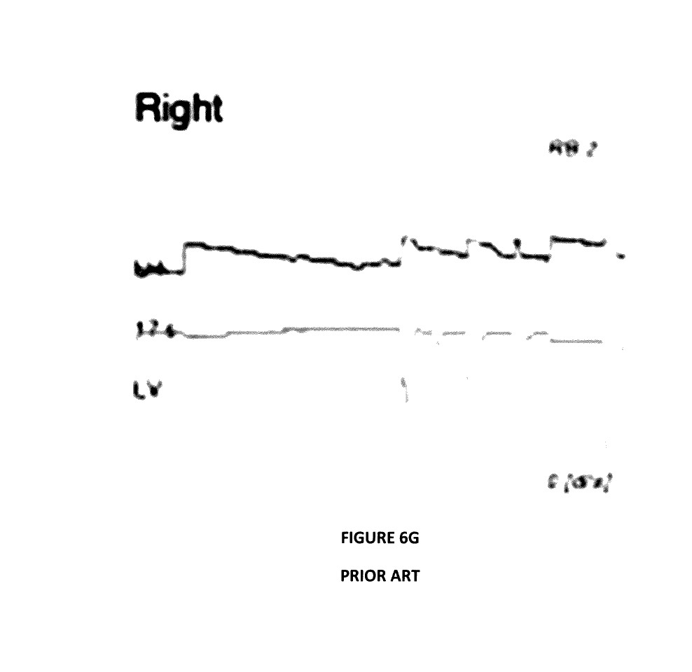

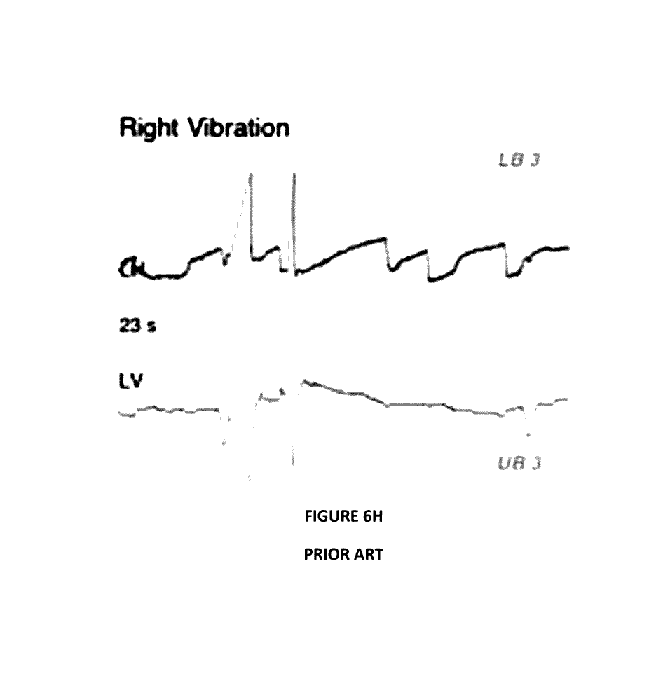

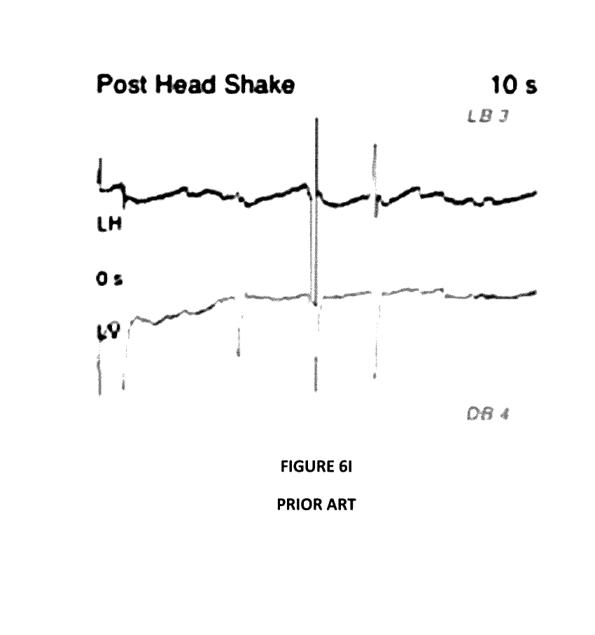

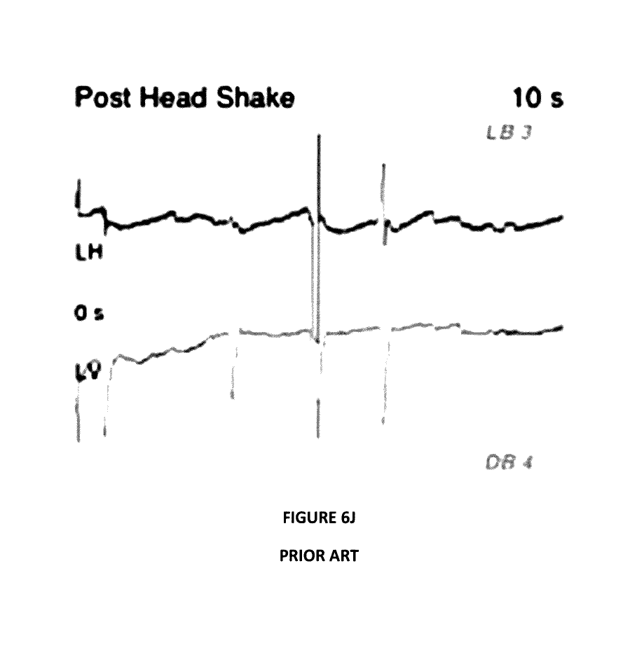

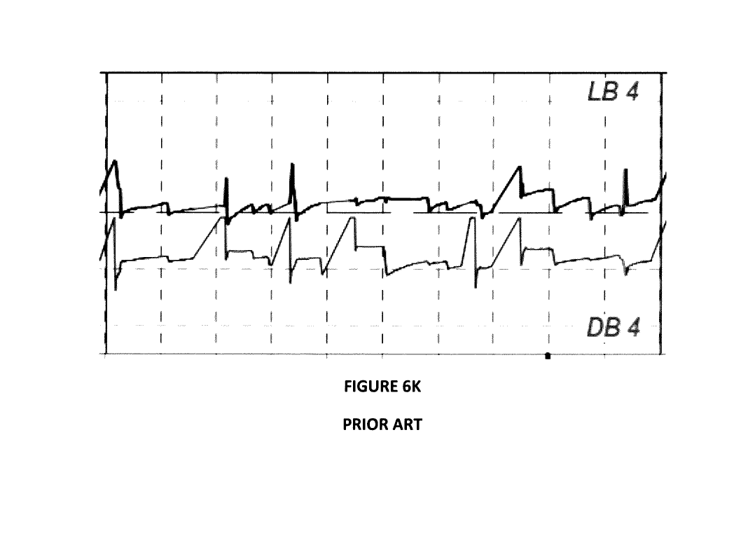

The “false positive” diagnostic errors resulting from the software's misinterpretation of eye blink artifact as nystagmus are not trivial. FIGS. 6A through 6K contain several examples. FIG. 6A shows that during vibration on the left side of the neck, eye blink artifact is analyzed by the computer as showing right beating and down beating nystagmus. This can be misinterpreted as representing a unilateral vestibular deficit, such as left vestibular neuritis. FIG. 6B shows that in the right Dix-Hallpike position, eye blink artifact is analyzed by the computer as showing right beating and up beating nystagmus. This can be misinterpreted as right posterior canal benign paroxysmal positional vertigo. FIG. 6C shows that in the right Dix-Hallpike position, eye blink artifact is analyzed by the computer as showing right beating nystagmus. This can be misinterpreted as lateral canal benign paroxysmal positional vertigo.

The screen shots in FIG. 6D show that in testing for rebound nystagmus, eye blink artifact is analyzed by the computer as showing left beating nystagmus. This can be misinterpreted as a cerebellar disorder. Similarly, the screen shots in FIG. 6E show that during vibration on either side of the neck, eye blink artifact is analyzed by the computer as showing left beating nystagmus. This can be misinterpreted as representing a unilateral vestibular deficit, such as right vestibular neuritis. As provided in FIG. 6F, during upright positional testing with head left, eye blink artifact in analyzed by the computer as showing left beating nystagmus. This can be misinterpreted as representing cervicogenic vertigo.

FIG. 6G illustrates that in testing for rebound nystagmus, eye blink artifact is analyzed by the computer as showing right beating nystagmus. This can be misinterpreted as a cerebellar disorder. FIG. 6H shows that during vibration on the right side of the neck, eye blink artifact is analyzed by the computer as showing left beating and up beating nystagmus. This can be misinterpreted as representing a unilateral vestibular deficit, such as right vestibular neuritis. FIG. 6I shows that after a head shaking maneuver, eye blink artifact is analyzed by the computer as showing left beating and down beating nystagmus. This can be misinterpreted as representing a unilateral vestibular deficit, such as right vestibular neuritis. FIG. 6J shows that after the Valsalva maneuver, eye blink artifact is analyzed by the computer as showing left beating nystagmus. This can be interpreted as representing a perilymphatic fistula or superior semicircular canal dehiscence. FIG. 6K shows that while sitting upright and trying to stare straight ahead, eye blink artifact is interpreted as showing down beating nystagmus. This can be interpreted as representing a cerebellar disorder.

It is therefore provided herein to improve upon and provide an algorithm or software component to assess the “degree of fitness” of the best-fit shape during image processing. It was found to base the determination of whether the eyelid is open or closed on whether the pupil was detectable; when the pupil is detectable the eyelid is open; when the pupil is undetectable the eyelid is closed. The pupil was chosen for this purpose because on grayscale images the greatest luminance differential is between the pupil and the iris, and since generally more of the pupil's perimeter is visible (as opposed to the iris, whose perimeter is usually partially occluded by the eyelids). It was therefore selected to not target the eyelids, because these can be fairly irregularly shaped, and because identification is often complicated by eye lashes or by makeup. For identification of the pupil it was decided to leverage simple properties of the Hough transform as provided for circle identification. The algorithm in accordance with the present invention is applied to each frame of an infrared video of eye movements as follows:
(a) the image is run through a kernel that serves as an edge-finding algorithm;
(b) the identified edges are run through an adjustable threshold algorithm to identify the most robust edges, since the luminance differential between any adjacent pixels tends to be greatest between the pupil (black) and the surrounding iris (light gray);
(c) several assumptions (based on the anatomy of the human eye) are made regarding the likely range of pupil diameters in a given video frame;
(d) the highest contrast edges are run through a Hough transform for circle identification, iterating from the smallest to the largest likely pupil diameter, rendering a Hough accumulator matrix;
(e) the amplitude of the peak (corresponding to the coordinates and diameter of the candidate circle most likely to represent the pupil) in the Hough accumulator matrix is compared to the average of the amplitudes of all other candidates in the matrix; this renders the absolute amplitude of the peak, as well as the average-to-peak ratio;
(f) those two results are compared to parameters that can be set by the user as thresholds for likelihood of pupil recognition; a larger “floor” of the absolute amplitude of the peak constitutes a more stringent criterion for pupil identification; a lower average-to-peak ratio constitutes a more stringent criterion for pupil identification. If the candidate circle meets the two criterion, then the algorithm judges a pupil to have been correctly identified, and the coordinates of its center are plotted for that frame on the videonystagmogram tracing. If the candidate circle fails to meet either of the two criterion, then the algorithm judges no pupil to have been identified, and no data are plotted for that frame on the videonystagmogram tracing.

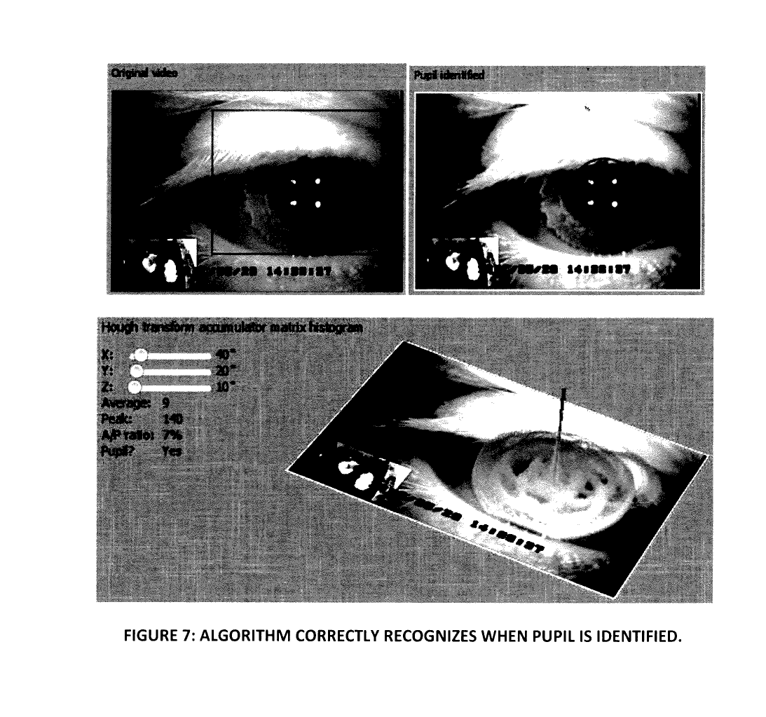

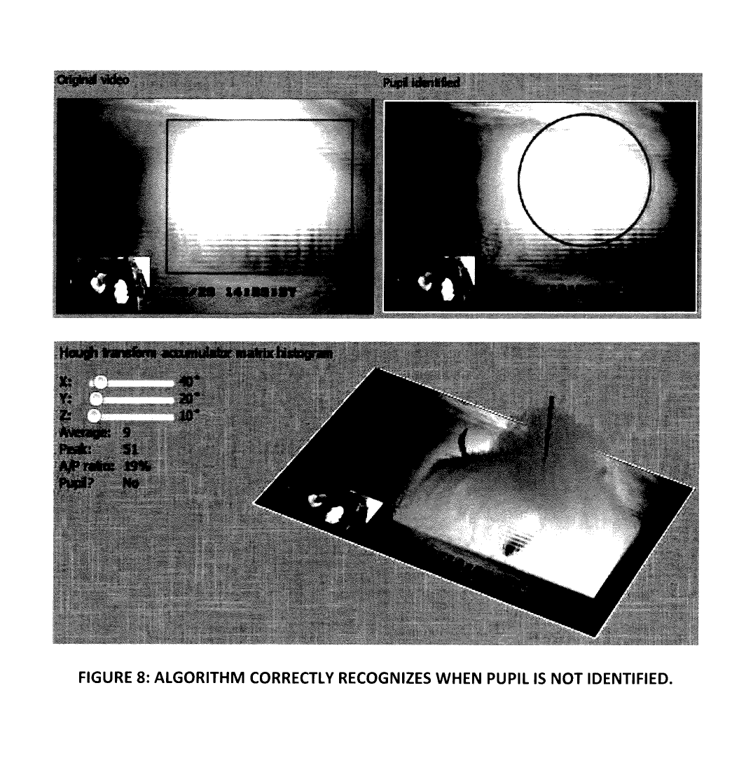

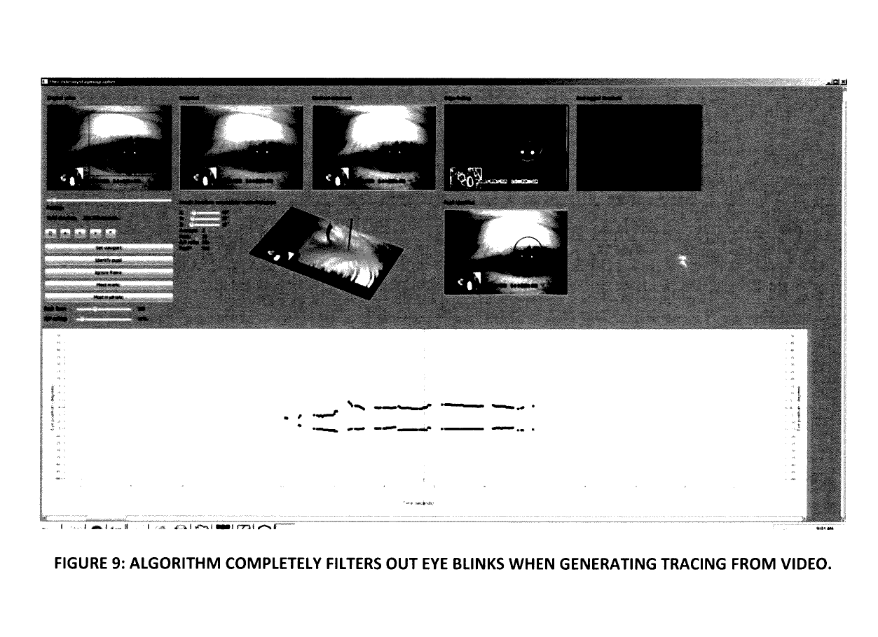

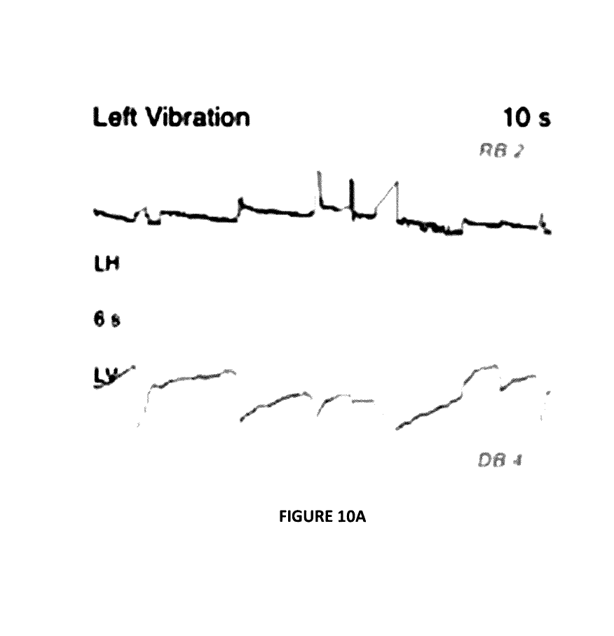

This algorithm robustly detects when a pupil is identified versus when no pupil is identified. In FIG. 7 is an example of correct recognition that a pupil has been identified. In FIG. 8 is an example of correct recognition that no pupil has been identified. When the algorithm is applied to a segment of video containing frequent eye blinks, the result is as shown in FIG. 9, resulting in the algorithm completely filtering out eye blinks when generating tracing from the video data. For the five seconds of eye movement video processed by this algorithm, a tracing is generated (bottom half of the screenshot). During the video, there are seven eye blinks, all of which are correctly filtered out by the new algorithm and appear simply as gaps in the tracing. This tracing is far “cleaner” than the examples cited earlier from a widely used commercially available package.

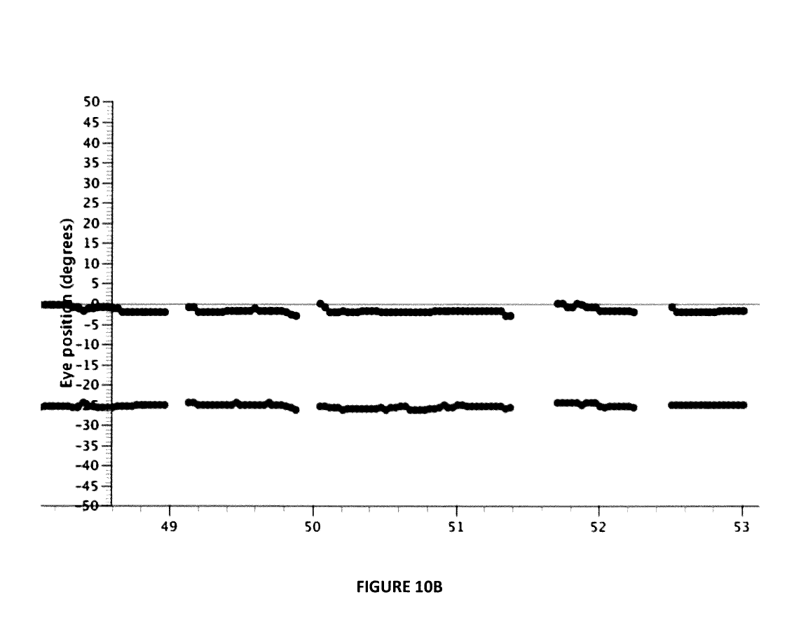

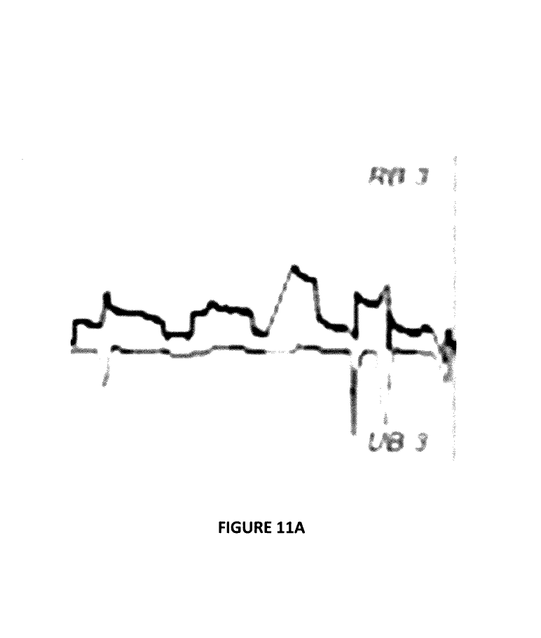

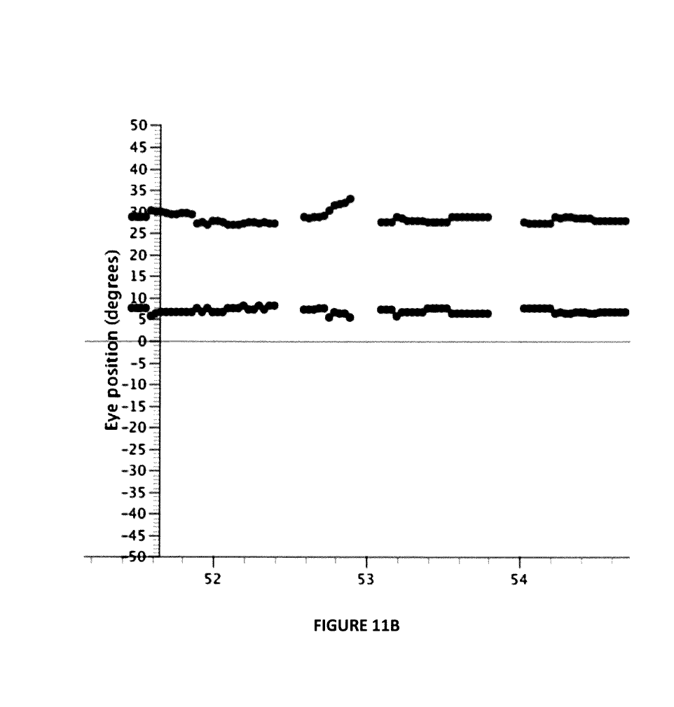

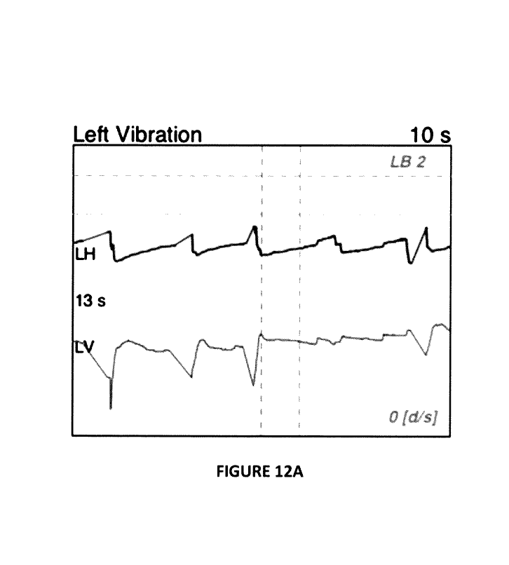

In FIGS. 10-19 below it is shown in comparison the performance of a widely used commercial software package such as Micromedical Spectrum with that of the algorithm in accordance with the present invention. In FIG. 10, while applying vibration on the left side of the neck, in the tracing on the left (Micromedical Spectrum), eye blinks are misinterpreted as right beating and down beating nystagmus, which would suggest a left sided vestibular deficit, such as vestibular neuritis. In the tracing on the right, the algorithm in accordance with the present invention, analysis of the same video eliminates the eye blinks entirely.

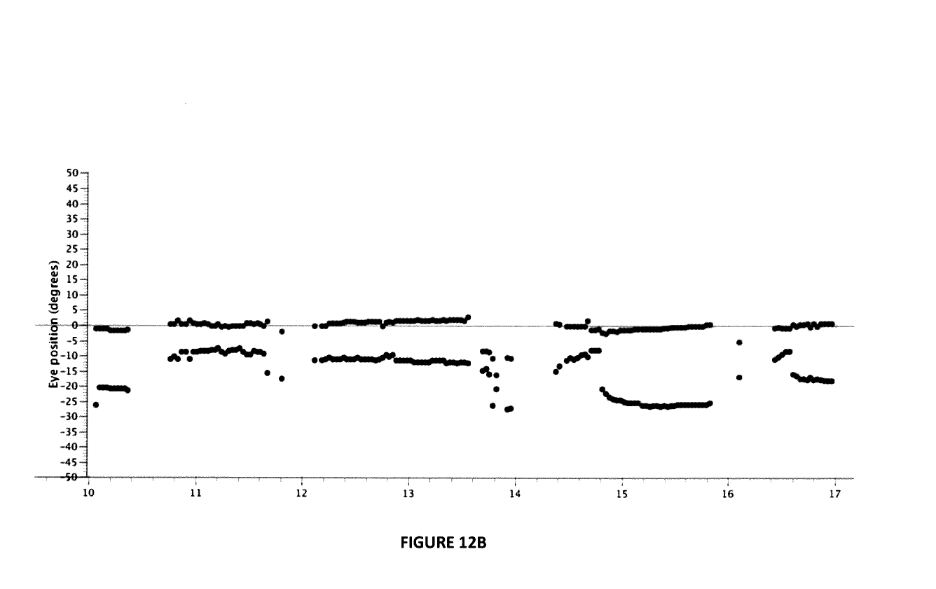

In FIG. 11, during the right Dix-Hallpike maneuver, in the tracing on the left (Micromedical Spectrum), eye blinks are misinterpreted as right beating and up beating nystagmus, which would suggest a diagnosis of right posterior canal benign paroxysmal positional vertigo. In the tracing on the right, the algorithm in accordance with the present invention, analysis of the same video eliminates the eye blinks entirely. In FIG. 12, during vibration on the left side of the neck, in the tracing on the left (Micromedical Spectrum), eye blinks are misinterpreted as left beating nystagmus, which would suggest a right sided vestibular deficit, such as vestibular neuritis. In the tracing on the right, the algorithm in accordance with the present invention, analysis of the same video eliminates the eye blinks entirely.

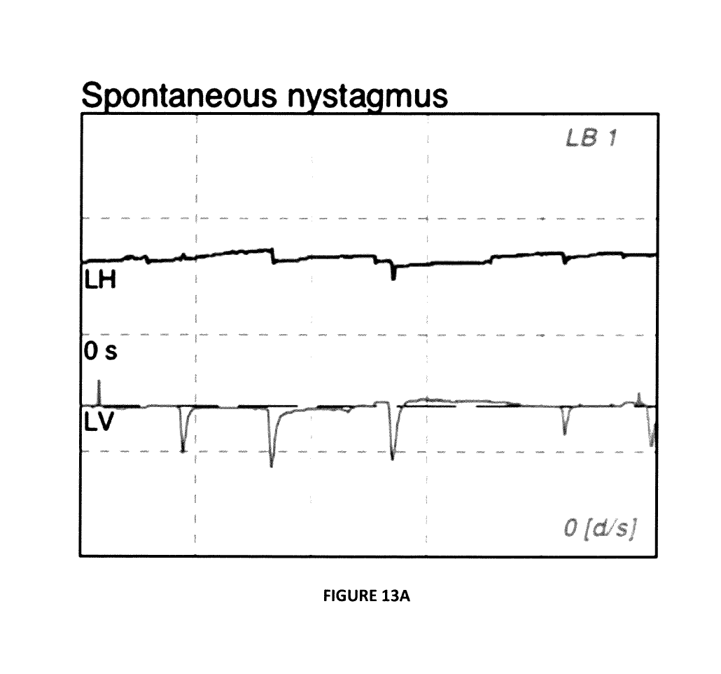

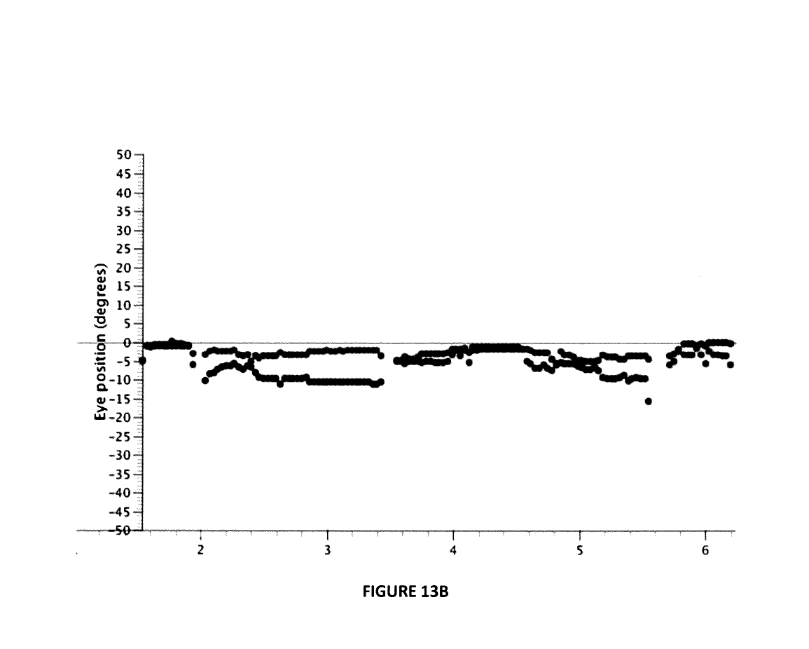

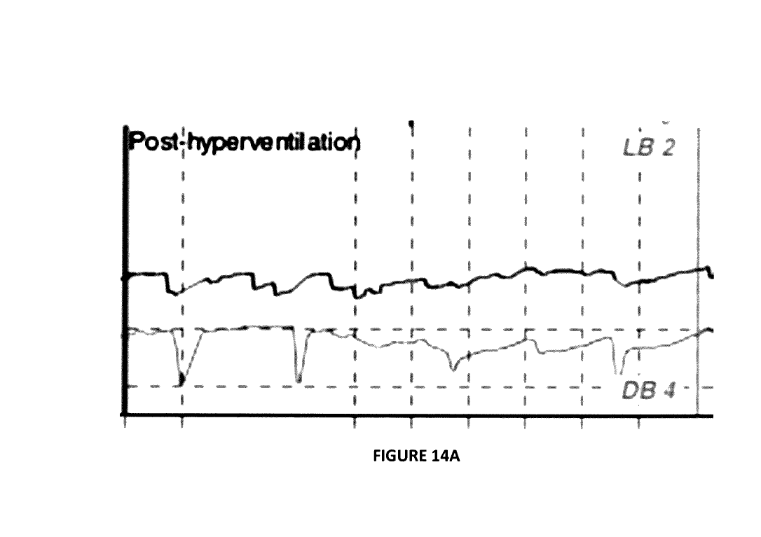

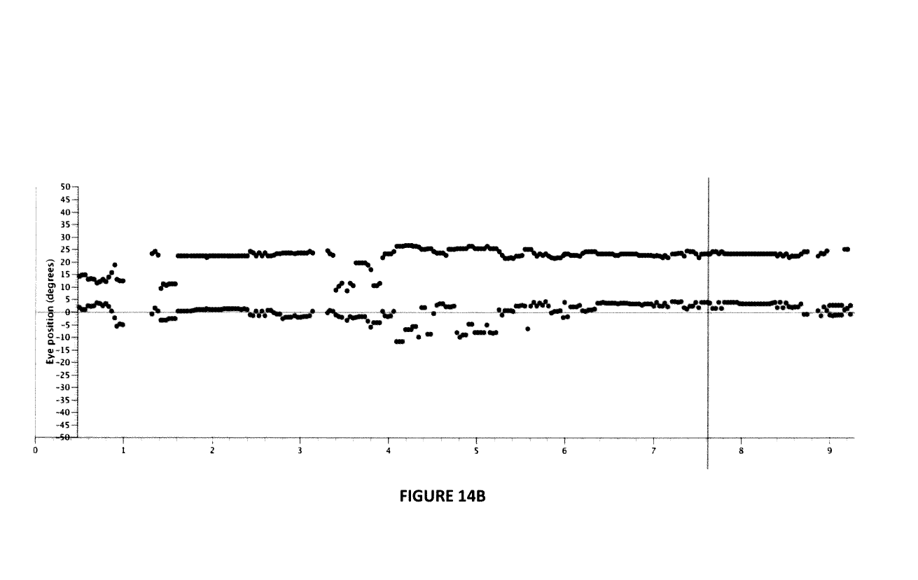

In FIG. 13, on primary position of gaze, in the tracing on the left (Micromedical Spectrum), eye blinks are misinterpreted as spontaneous left beating nystagmus, which would suggest a right sided vestibular deficit, such as vestibular neuritis. In the tracing on the right, the algorithm in accordance with the present invention, analysis of the same video eliminates the eye blinks entirely. In FIG. 14, after hyperventilation, in the tracing on the left (Micromedical Spectrum), eye blinks are misinterpreted as left beating and down beating nystagmus, which could suggest a left sided lesion such as a vestibular schwannoma. In the tracing on the right, the algorithm in accordance with the present invention, analysis of the same video eliminates the eye blinks.

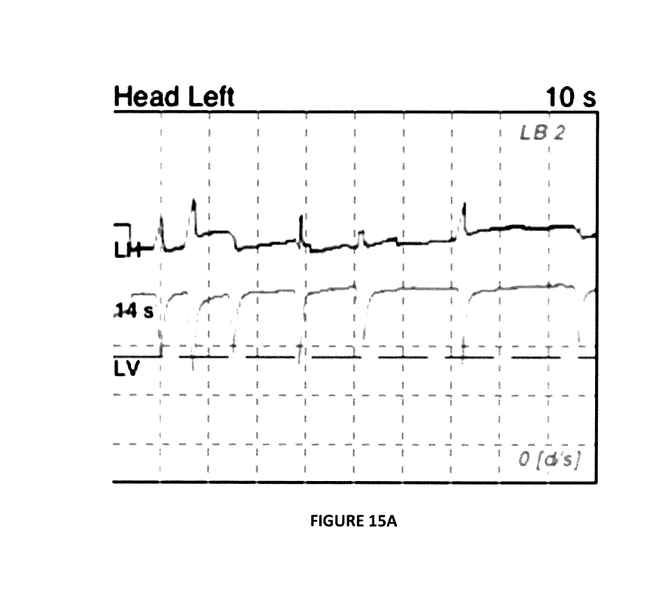

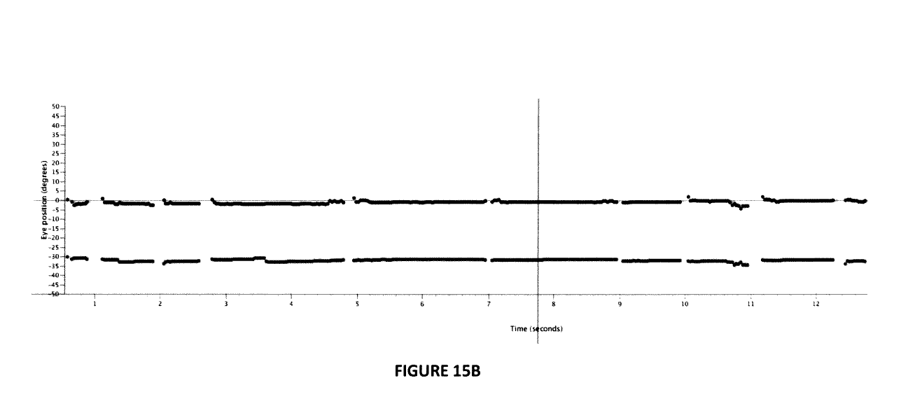

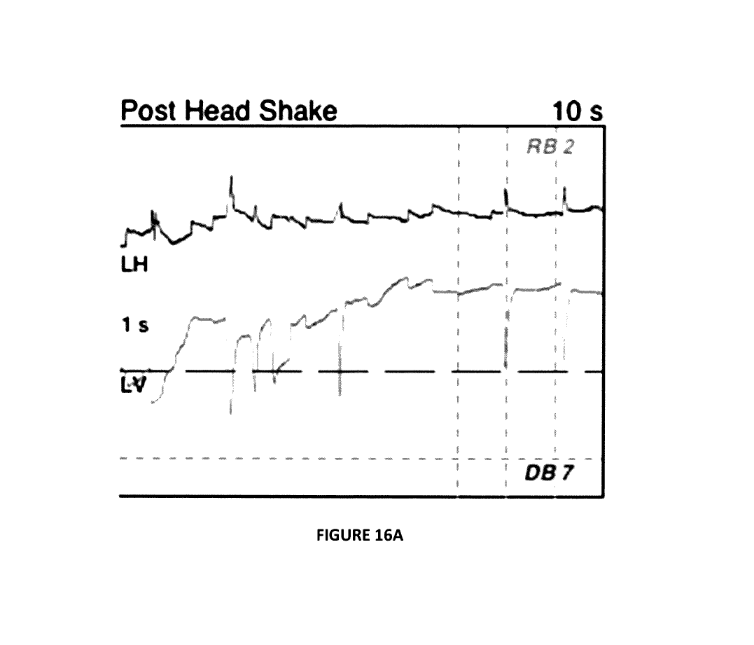

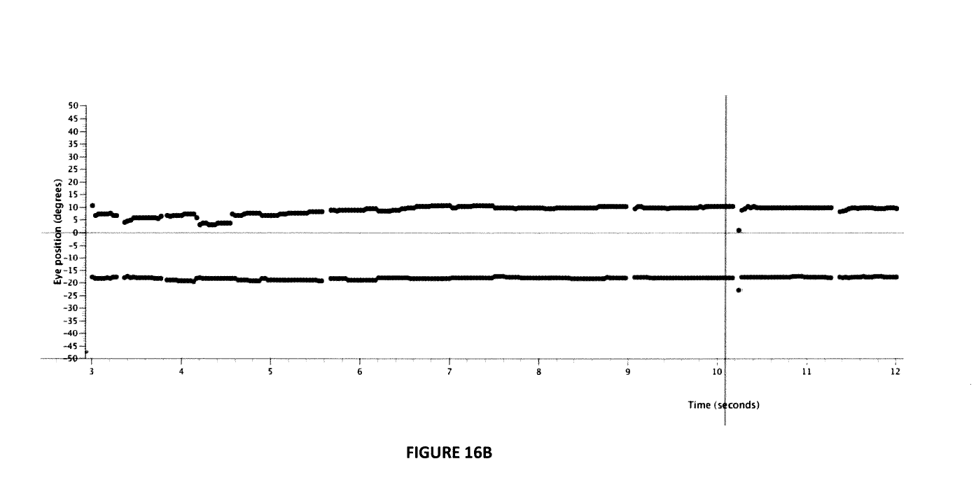

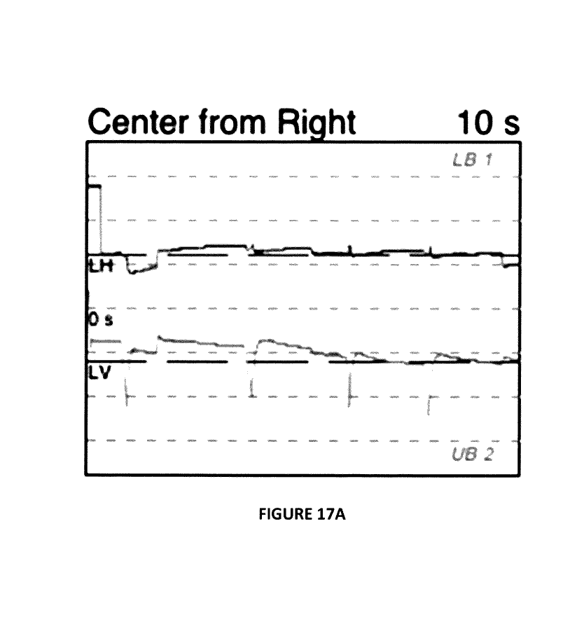

In FIG. 15, in the upright head left position, in the tracing on the left (Micromedical Spectrum), eye blinks are misinterpreted as left beating nystagmus, which could suggest a cervical lesion. In the tracing on the right, the algorithm in accordance with the present invention, analysis of the same video eliminates the eye blinks entirely. In FIG. 16, after head shaking, in the tracing on the left (Micromedical Spectrum), eye blinks are misinterpreted as right beating and down beating nystagmus, which would suggest a left sided vestibular deficit, such as vestibular neuritis. In the tracing on the right, the algorithm in accordance with the present invention, eliminates the eye blinks entirely.

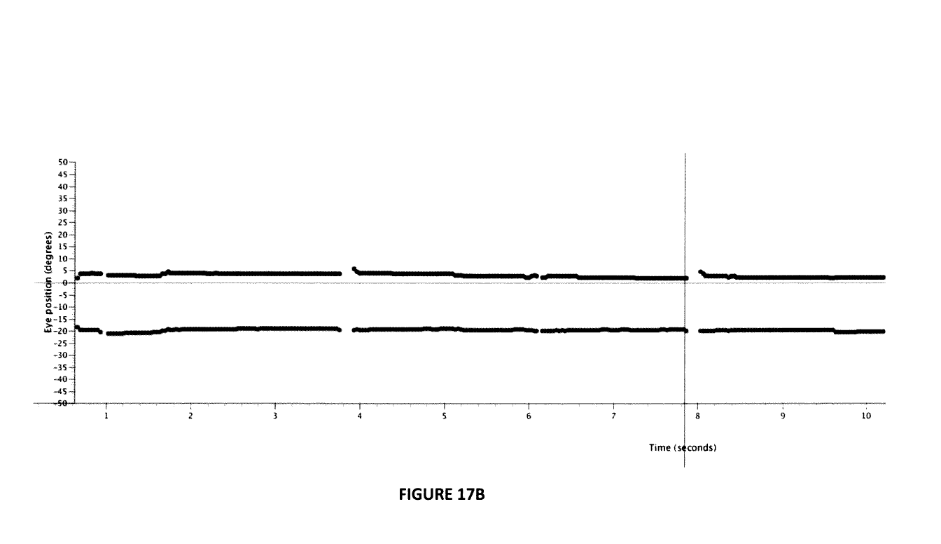

In FIG. 17, after prolonged rightward gaze the patient returns to primary position of gaze. In the tracing on the left (Micromedical Spectrum), eye blinks are misinterpreted as left beating and up beating nystagmus, which could suggest a cerebellar lesion. In the tracing on the right, the algorithm in accordance with the present invention, eliminates the eye blinks entirely.

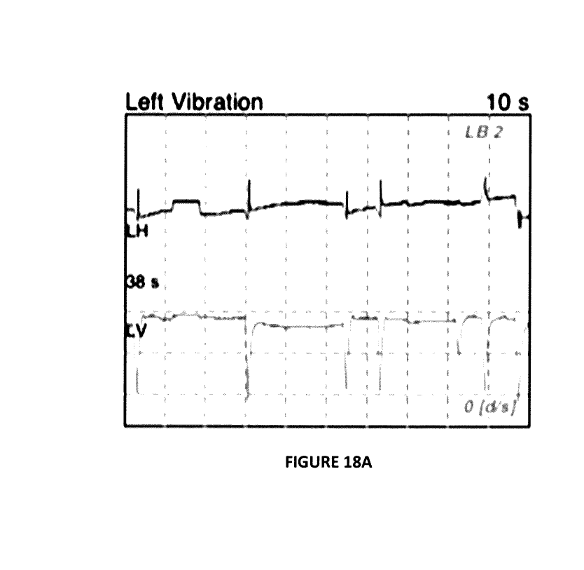

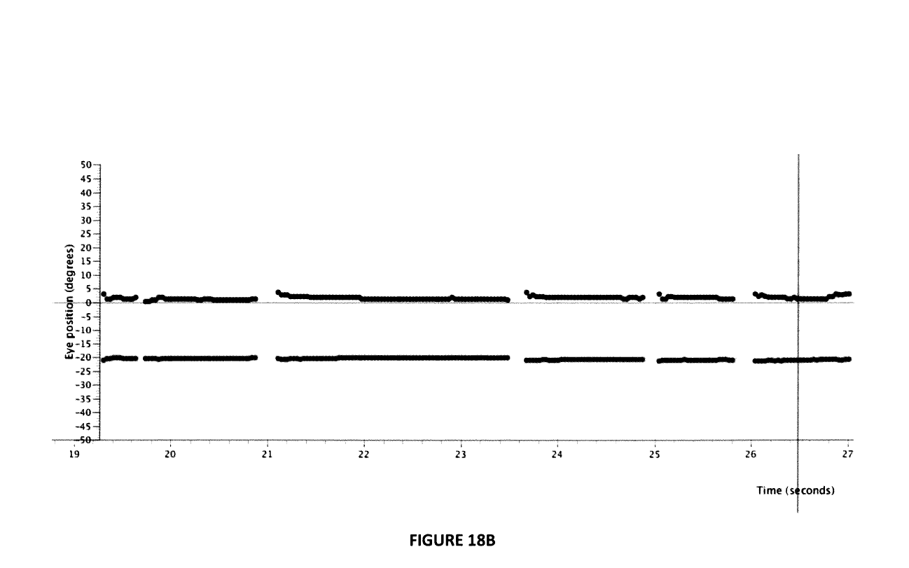

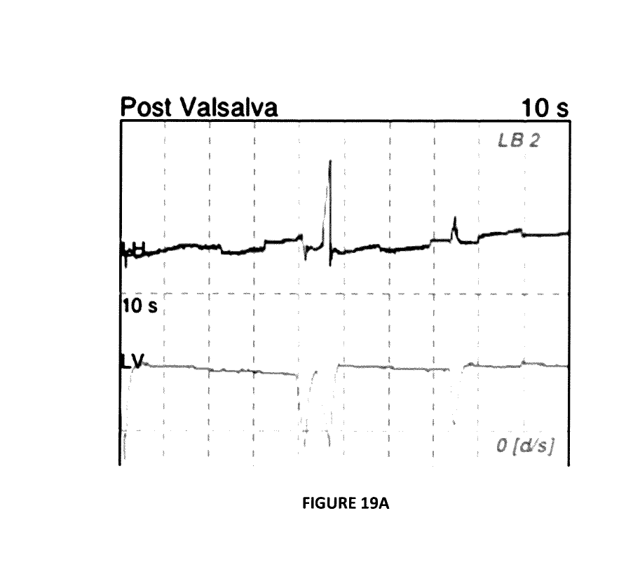

In FIG. 18, during vibration on the left side of the neck, in the tracing on the left (Micromedical Spectrum), eye blinks are misinterpreted as left beating nystagmus, which would suggest a right sided vestibular deficit, such as vestibular neuritis. In the tracing on the right, the algorithm in accordance with the present invention, eliminates the eye blinks entirely. In FIG. 19, during Valsalva maneuver, in the tracing on the left (Micromedical Spectrum), eye blinks are misinterpreted as left beating nystagmus, which could suggest superior semicircular canal dehiscence or a perilymphatic fistula. In the tracing on the right, the algorithm in accordance with the present invention, eliminates the eye blinks entirely.

As provided in accordance with the present invention there is provided a simple, effective algorithm for filtering out eye blink artifact at the level of individual grayscale images commonly acquired for medical diagnostic purposes by infrared videonystagmography. Similar to some previous approaches, the current one employs shape recognition by a Hough transform; however, the novelty of the present approach is that we leverage simple properties of the Hough transform's accumulator matrix to determine “goodness of fit” of the shape recognition, where “poor fit” corresponds to correct recognition that no pupil has been identified—and when the pupil has not been identified, no data are plotted on the videonystagmographic tracing. By generating a cleaner tracing, this approach will facilitate computerized interpretation of the tracing, and in doing so, would aid the non-subspecialist physician in diagnosis.

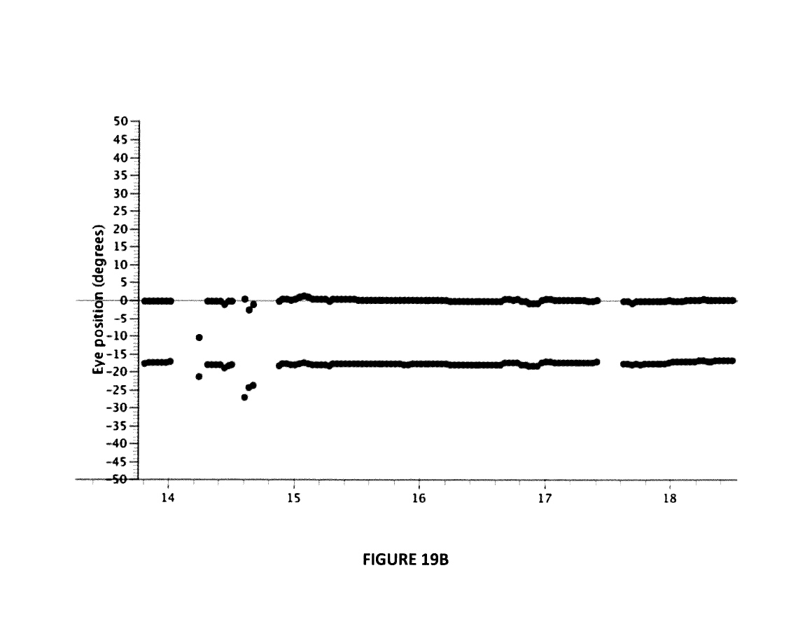

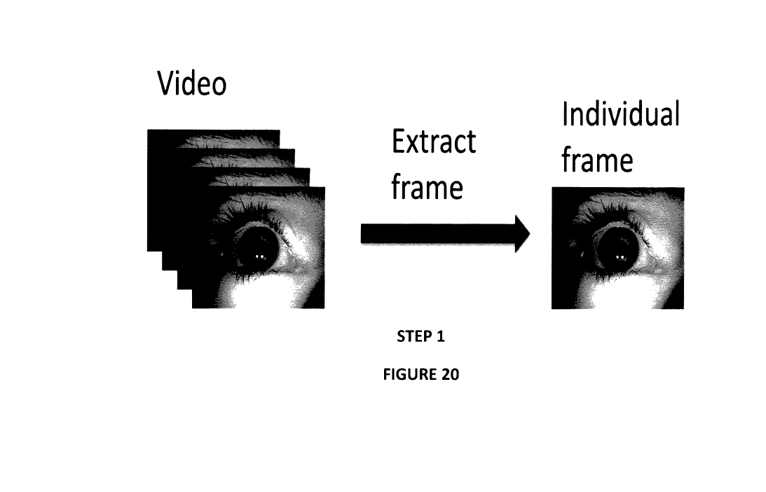

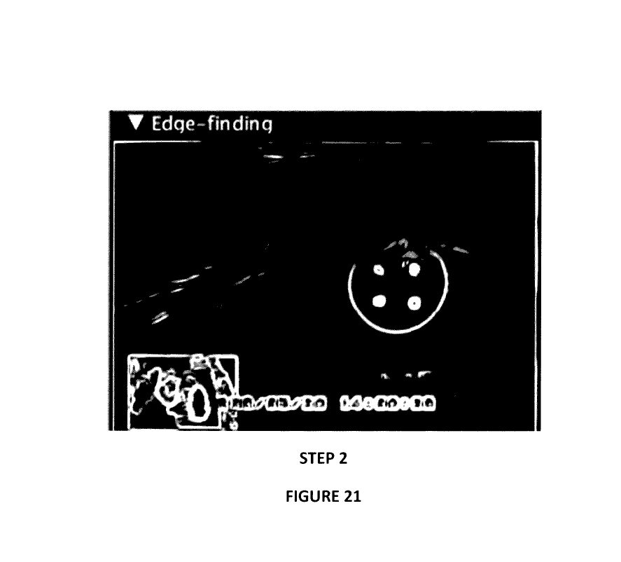

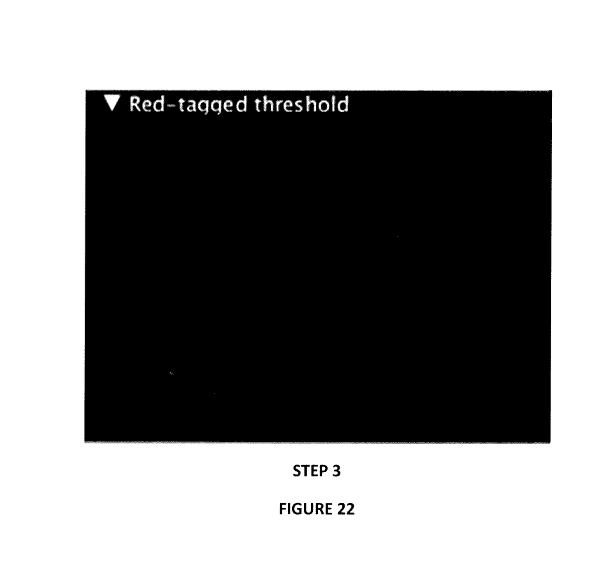

The algorithm software component is applied to an infrared video stream of the movement of a single eye in the following method. In Step 1 (FIG. 20), a grayscale image frame is extracted from the video stream. In Step 2 (FIG. 21), the image is run through a kernel that serves as an edge-finding algorithm. A variety of edge-finding algorithms can be employed for this, such as Canny edge detection. In Step 3 (FIG. 22), the identified edges are run through an adjustable threshold algorithm (that, at the user's discretion, can be manually calibrated) to identify the most robust edges, since the luminance differential between any adjacent pixels tends to be greatest between the pupil (black) and the surrounding iris (light gray).

In Step 4, several assumptions are made regarding the likely range of pupil diameters in a given video frame. The first set of assumptions derives from the anatomy of the human eye. The second set of assumptions derives from whether any pupil has been correctly identified in the preceding few milliseconds; the pupil (if present) of the current frame should be relatively close in diameter (to the most recently identified pupil), since the maximum rate of change in pupil diameter is relatively slow. The purpose of these assumptions is to limit the range of diameters through which to search, thereby also limiting computational burden in order to make the algorithm more efficient.

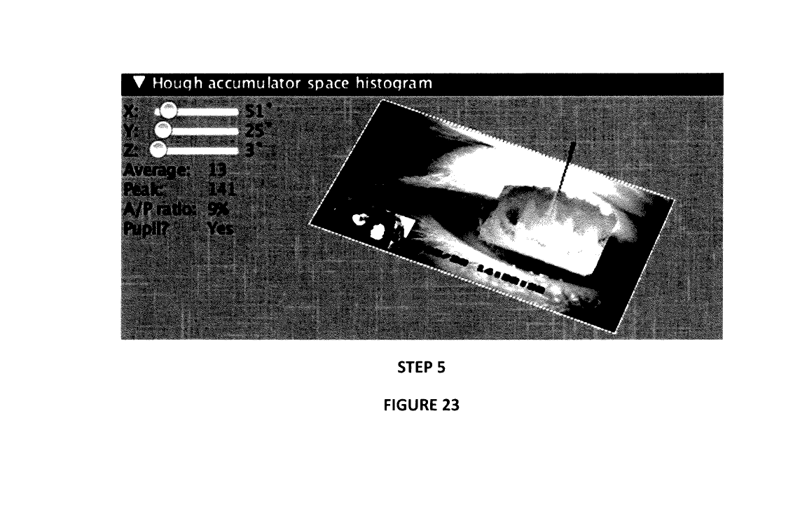

In Step 5 (FIG. 23), the highest contrast edges are run through a Hough transform for circle identification, iterating from the smallest likely pupil diameter to the largest likely pupil diameter, rendering a Hough accumulator matrix. In another embodiment, a Hough transform for ellipse identification would be applied (since when the direction of regard is very oblique—i.e., the eye is not aimed directly at the camera—the pupil will appear more elliptical than circular). In Step 6, the amplitude of the identified peak (corresponding to the coordinates of the center and diameter of the candidate circle most likely to represent the pupil) in the Hough accumulator matrix is compared to the average of the amplitudes of all other candidates in the matrix; this renders the absolute amplitude of the peak, as well as the average-to-peak ratio.

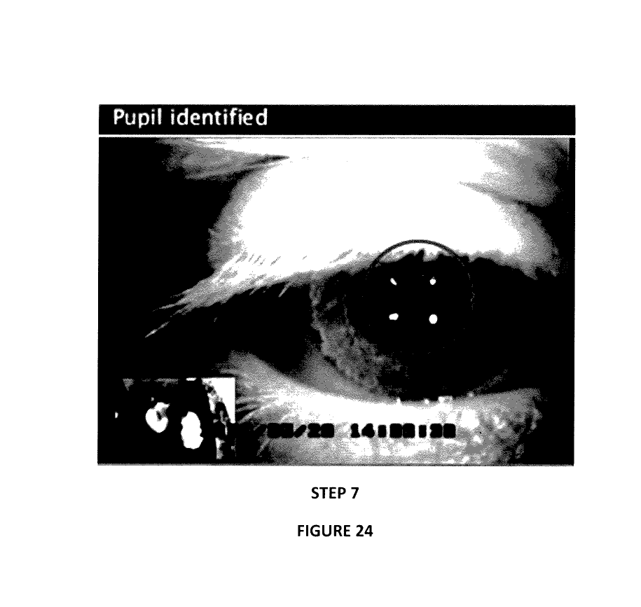

In Step 7 (FIG. 24), those two results (the amplitude of the peak, and the average-to-peak amplitude) are compared to two parameters comprising threshold criterion for likelihood of pupil recognition; these parameters can, at the user's discretion, be manually calibrated. The first parameter is the “floor” (the lowest acceptable value) of the absolute amplitude of the peak of the candidate circle; a larger “floor” constitutes a more stringent criterion for pupil identification. The second parameter is the “average-to-peak ratio,” which quantifies the degree to which a candidate circle is a “better-fit circle” than all the other candidate circles; a lower average-to-peak ratio constitutes a more stringent criterion for pupil identification. If the candidate circle meets the two criterion, then the algorithm judges a pupil to have been correctly identified, and the Cartesian coordinates of its center are plotted (as a function of time) for that frame on the videonystagmogram tracing. If the candidate circle fails to meet either of the two criterion, then the algorithm judges no pupil to have been identified, and no data are plotted for that frame on the videonystagmogram tracing.

In Step 8, the process returns to Step 1 to advancing to the next frame for extraction until the end of the video stream.

The above system process steps can be either defined to run in a system or defined as a method for processing the various steps. Both of which are covered by the present invention.

From the foregoing and as mentioned above, it is observed that numerous variations and modifications may be effected without departing from the spirit and scope of the novel concept of the invention. It is to be understood that no limitation with respect to the embodiments illustrated herein is intended or should be inferred. It is intended to cover, by the appended claims, all such modifications within the scope of the appended claims.

I claim:

1. In a system for videonystagmography (VNG) testing of a pupil in an eye that records oculomotor response data and has a computing device configured with software to determine and display on a display device a plot representation of the correlated data, comprising an improvement to software instructions that determine pupil recognition and plots representation of the correlated data, said software instructions being further:
configured to extract a grayscale image from a frame in the recorded oculomotor response data;
configured to locate and to identify an edge of a shape from the extracted image of the eye, referred to as an identified shape edge;
configured to determine a center and diameter from the identified shape edge and to store the diameter in a range from smallest to largest probable diameters;
configured to run a shape identification Hough transform on the identified shape edge to identify a shape representing a candidate pupil in the extracted image, wherein the Hough transform iterates from the smallest probable diameter to the largest probable diameter and the software is further configured to render an accumulator matrix;
configured to compare a current amplitude of the center and the diameter of the candidate shape to an average of amplitudes of other candidate shapes defined by the accumulator matrix to define an absolute amplitude of the center and the diameter and to define an average-to-peak ratio;
configured to compare the absolute amplitude and the average-to-peak ratio to two threshold criterion parameters to determine the likelihood of pupil recognition;
configured to plot coordinates representation of the center of the shape only when the shape meets the two threshold criterion parameters for pupil recognition.

2. The system of claim 1, wherein the software is further configured to identify an adjusted edge shape from the identified shape edge based on an adjustable threshold algorithm.

3. The system of claim 2, wherein the software is further configured to compare the adjusted shape edge in the extracted image to a previous identified shape edge in a previous extracted image to determine the shape diameter.

4. The system of claim 3, wherein the software is further configured to run the shape identification Hough transform on the adjusted shape edge to identify the candidate shape and to render the Hough accumulator matrix.

5. The system of claim 1, wherein the software is configured to advance to the subsequent frame in the recorded oculomotor response data without plotting the center of the shape coordinates when the candidate shape fails to meet the two threshold criterion parameters for pupil recognition.

6. The system of claim 1, wherein the software is further configured to repeat for each frame in the recorded oculomotor response data.

7. The system of claim 1, wherein the shape identification transform is based on a circular identification transform.

8. The system of claim 1, wherein the shape identification transform is based on an elliptical identification transform.

9. The system of claim 1, wherein a first threshold criterion parameter is an absolute amplitude of the peak of the candidate shape and is configured to a low acceptable value to define a less stringent criterion for pupil identification or configured to a high acceptable value to define a more stringent criterion for pupil identification.

10. The system of claim 1, wherein a second threshold criterion parameter is the average-to-peak ratio and is configured as a best-fit-circle to quantify a candidate shape and wherein a low average-to-peak ratio is configured for a more stringent criterion for pupil identification and wherein a high average-to-peak ratio is configured for a less stringent criterion for pupil identification.

11. The system of claim 1, wherein the candidate shape is either a circle or ellipse.

12. In a method for videonystagmography (VNG) testing a pupil in an eye that records oculomotor response data and has a computing device configured with software to determine and display on a display device a plot representation of the correlated data, the method comprising an improvement to software instructions that determine pupil recognition and plots representation of the correlated data, said software instructions configured for:
extracting a grayscale image from a frame in the recorded oculomotor response data;
locating and identifying an edge of a shape from the extracted image of the eye, referred to as an identified shape edge;
determining a center and a diameter from the identified shape edge and storing the diameter in a range from smallest to largest probable diameters;
running a shape identification Hough transform on the identified shape edge and identifying a shape representing a candidate pupil in the extracted image, wherein the Hough transform iterates from the smallest probable diameter to the largest probable diameter and the software is configured to rendering an accumulator matrix;
comparing a current amplitude of the center and the diameter of the candidate shape to an average of amplitudes of other shapes defined by the accumulator matrix for defining an absolute amplitude of the center and the diameter and for defining an average-to-peak ratio;
comparing the absolute amplitude and the average-to-peak ratio to two threshold criterion parameters for determining the likelihood of pupil recognition;
plotting coordinates representing the center of the shape only when the shape meets the two threshold criterion parameters for pupil recognition.

13. The system of claim 12, wherein the software is further configured for identifying an adjusted edge shape from the identified shape edge based on an adjustable threshold algorithm.

14. The system of claim 13, wherein the software is further configured for comparing the adjusted shape edge in the extracted image to a previous identified shape edge in a previous extracted image to determine the shape diameter.

15. The system of claim 14, wherein the software is further configured for running the shape identification Hough transform on the adjusted shape edge and for identifying the candidate shape and rendering the Hough accumulator matrix thereon.

16. The system of claim 12, wherein the software is configured for advancing to a subsequent frame in the recorded oculomotor response data without plotting the center of the shape coordinates when the candidate shape fails to meet the two threshold criterion parameters for pupil recognition.

17. The system of claim 12, wherein the software is further configured for repeating the process for each frame in the recorded oculomotor response data.

18. The system of claim 12, wherein the shape identification transform is based on a circular identification transform.

19. The system of claim 12, wherein the shape identification transform is based on an elliptical identification transform.

20. The system of claim 12, wherein a first threshold criterion parameter is an absolute amplitude of the peak of the candidate shape and is configured to a low acceptable value to define a less stringent criterion for pupil identification or configured to a high acceptable value to define a more stringent criterion for pupil identification.

21. The system of claim 12, wherein a second threshold criterion parameter is the average-to-peak ratio and is configured as a best-fit-circle to quantify a candidate shape and wherein a low average-to-peak ratio is configured for a more stringent criterion for pupil identification and wherein a high average-to-peak ratio is configured for a less stringent criterion for pupil identification.

22. The system of claim 12, wherein the candidate shape is either a circle or ellipse.
